(19) United States
(12) Patent Application Publication
(10) Pub. No.: US 2007/0123917 A1
(43) Pub. Date: May 31, 2007

(54) ANASTOMOTIC DEVICE PROMOTING TISSUE NECROSIS

(76) Inventors: Mark S. Ortiz, Milford, OH (US); Robert Jason Simms, Liberty Township, OH (US); Tom Allen Ditter, San Ramon, CA (US); David Nicolas Plescia, Cincinnati, OH (US); Soane H. Eke, Palo Alto, CA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 11/164,574

(22) Filed: Nov. 29, 2005

(51) Int. Cl. A61B 17/08

(52) U.S. Cl. 606/153

(57) ABSTRACT

Embodiments of the present invention generally provide an anastomosis device that can be used to couple two or more layers of tissue in apposition. In an exemplary embodiment, the device can be formed from one or more woven wires that can be configured to have a generally tubular shape in a first, expanded position for insertion to an anastomosis site and a generally annular, ring-shape in a second, resting position for securing tissue in apposition at the anastomosis site. The device can be adapted either to cause necrosis of the tissue layers around an outer periphery of the device or to promote growth of tissue about an inner periphery of the device.

### FIELD OF THE INVENTION

The invention relates broadly to an anastomosis device, and more particularly to an anastomosis device formed from a woven tube of shape-memory wire where, when deployed, the anastomosis device can hold tissues of an anastomotic site in apposition.

### BACKGROUND OF THE INVENTION

During many surgical procedures, the surgeon will have to close or ligate various blood vessels and other ducts before severing them in order to prevent excessive bleeding, and reduce the risk of other complications to the patient. One ligation technique is to tie a suture about the vessel to close the vessel. Alternatively, a surgeon can place a clip having a pair of legs connected at their proximal ends about the vessel, and urge or squeeze the legs together to close the vessel.

One drawback associated with some current clips used for ligating vessels is that the legs of the clip may tend to separate to some extent following release from a clip applier. This phenomenon is called duck-billing. Duck-billing can result in insufficient ligation of a vessel, thus leading to excessive blood loss and/or unnecessary damage to the vessel. Further, some known ligation clips are often difficult to preload into a clip applier because of resistance between the tissue disposed between the jaws and the gripping features on the clip legs.

Accordingly, there remains a need for an improved surgical instrument and method, and in particular for surgical clips used for ligating blood vessels, other ducts, and the like.

### BRIEF SUMMARY OF THE INVENTION

Embodiments of the present invention generally provide anastomosis devices for securing layers of tissue, such as the walls of a small intestine and an upper stomach pouch, in apposition. An anastomosis device, in one embodiment of the invention, generally includes a body that can be formed from a woven shape-memory wire having a central lumen. The body can be configurable between an expanded position, in which the body can assume a generally tubular configuration having a wire mesh wall adapted for insertion into a lumen of an anastomotic site, and a rest position, in which the body can assume an annular configuration having a plurality of petals with petal tips that define an outer periphery. The rest position is effective to hold opposed tissues of the anastomotic site in apposition and can apply a pressure to the opposed tissues such that the pressure decreases from an outer periphery to an inner periphery of the device. The plurality of petals can be configured to apply the pressure to the opposed tissues to cause necrosis of the opposed tissues in a contact region, such as a region about the outer periphery of the device. In one embodiment, in the rest position, the tips of the petals are adapted to contact the opposed tissues along a distance greater than approximately 5% of the circumference of the outer periphery of the device.

In one embodiment of the anastomosis device, the plurality of petals can include a superior set of adjacent petals and an inferior set of adjacent petals where the device is adapted to receive the opposed tissues between the inferior and superior sets of petals. In a rest position, each petal of the superior set and inferior set of adjacent petals can be formed by adjacent arms connected by a tip that extends along a portion of the outer periphery of the device. Each petal tip can have a first radius and each arm can be connected to the tip at a bend having a second radius, where the first radius is greater than the second radius. The tips of the superior set of adjacent petals have midpoints that can be staggered about a circumference of the device relative to midpoints of the tips of the inferior set of adjacent petals or that can be substantially aligned with midpoints of the tips of the inferior set of adjacent petals.

The superior and inferior petals of the device can be configured with a variety of geometries. In one embodiment, when the device is configured in the rest position, the adjacent arms and tip of each petal can be oriented in a substantially planar configuration in a plane that is substantially perpendicular to a central axis extending through the central lumen of the device. In another embodiment, the adjacent arms of each petal can be oriented in a plane that is substantially perpendicular to a central axis extending through the central lumen of the device where at least a portion of the adjacent arms of the superior set of petals include an arc portion having an inferior facing opening, and at least a portion of the adjacent arms of the inferior set of petals include an arc portion having a superior facing opening. The adjacent arms of the superior set of petals can include a bend portion disposed between the arc portion and the tip that can orient the tip in the plane that is substantially perpendicular to a central axis extending through the central lumen of the device and the adjacent arms of the inferior set of petals can include a bend portion disposed between the arc portion and the tip. The bend portion of the adjacent arms can orient the tip in the plane that is substantially perpendicular to a central axis extending through the central lumen of the device. In yet another embodiment, the tip of each petal can have a peak portion that defines an inferior facing opening and a trough portion that defines a superior facing opening.

In another embodiment, the anastomosis device can include a first wire circumferentially coupled to the petal tips of the superior set of adjacent petals and a second wire circumferentially coupled to the petal tips of the inferior set of adjacent petals. When the device is configured in the rest position, the first wire and the second wire can contact the opposed tissues along a distance greater than approximately 90% of the circumference of the outer periphery of the device.

Methods for coupling tissue layers are also provided. In one embodiment, the method can include delivering an anastomosis device, formed from a woven shape-memory wire having a wire mesh wall defining a central lumen and configured in an expanded, tubular position, through a lumen defined by two opposed tissues. The method can also include deploying the anastomosis device to hold the opposed tissues in apposition such that, upon deployment, the device assumes an annular configuration having a plurality of petals with petal tips that define an outer periphery. As so deployed, the device is effective to apply a pressure to the opposed tissues such that the pressure decreases from the outer periphery to an inner periphery of the device.

### BRIEF DESCRIPTION OF THE DRAWINGS

The invention will be more fully understood from the following detailed description taken in conjunction with the accompanying drawings, in which:

FIG. 1 is a top view of a prior art anastomosis device shown in a deployed state;

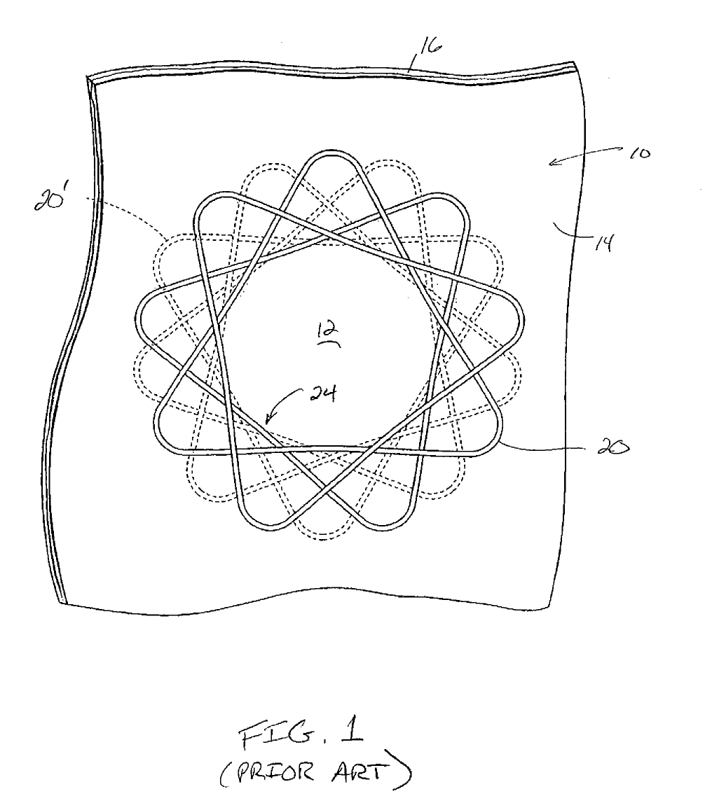

FIG. 2 is a side view of one embodiment of a tissue-necrosing type anastomosis device in an expanded elongate tubular configuration;

FIG. 3A. is a top view of one embodiment of a tissue-necrosing type anastomosis device in a deployed state having staggered petals with non-overlapping tips;

FIG. 3B is a side view of the anastomosis device of FIG. 3A;

FIG. 3C is a perspective view of the anastomosis device of FIG. 3A having a tissue material disposed between a portion of the opposed petals;

FIG. 4A is a top view of one embodiment of a tissue-necrosing type anastomosis device in a deployed state having staggered overlapping petals;

FIG. 4B is a side view of the tissue-necrosing type anastomosis device of FIG. 4A;

FIG. 4C is a perspective view of the embodiment of the tissue-necrosing type anastomosis device of FIG. 4A having a tissue material disposed between a portion of the opposed petals;

FIG. 4D is a side view of another embodiment of the tissue-necrosing type anastomosis device of FIG. 4A;

FIG. 5A is a top view of one embodiment of a tissue-necrosing type anastomosis device in a deployed state having staggered overlapping petals;

FIG. 5B is a side view of the anastomosis device of FIG. 5A;

FIG. 5C is a perspective view of the anastomosis device of FIG. 5A having a tissue material disposed between a portion of the opposed petals;

FIG. 6A is one embodiment of a tissue-necrosing type anastomosis device in a deployed state having aligned overlapping petals;

FIG. 6B is a side view of the anastomosis device of FIG. 6A;

FIG. 6C is a perspective view of the anastomosis device of FIG. 6A having a tissue material disposed between a portion of the opposed petals;

FIG. 7 illustrates an embodiment of a tissue-necrosing type anastomosis device in a deployed state having a pressure distribution ring disposed about the tips of the petals;

FIG. 8 illustrates the anastomosis device of FIG. 7 prior to formation of the pressure distribution rings;

FIG. 9A is a top view of one embodiment of an anastomosis device configured to promote growth of tissue about an inner periphery of the device;

FIG. 9B is a perspective view of the anastomosis device of FIG. 9A; and

FIG. 9C is a perspective view of the anastomosis device of FIG. 9A deployed at an anastomosis site.

### DETAILED DESCRIPTION OF THE INVENTION

Certain exemplary embodiments will now be described to provide an overall understanding of the principles, structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with features of other embodiments. Such modifications and variations are intended to be included within the scope of the present invention.

The present invention generally provides an anastomosis device that can be used to couple two or more layers of tissue in apposition. In an exemplary embodiment, the device can be formed from one or more woven wires that can be configured to have a generally tubular shape in a first, expanded position for insertion to an anastomosis site and a generally annular, ring-shape in a second, resting or deployed position for securing tissue in apposition at the anastomosis site. The device can be adapted either to cause necrosis of the tissue layers around an outer periphery of the device or to promote growth of tissue about an inner periphery of the device.

The device can be formed from a variety of materials, but in an exemplary embodiment, it is formed from a shape-memory wire woven into a mesh. The shape-memory wire is formed into an annular ring such that, when the anastomosis device is expanded into an elongate tubular form, it will return to its annular, ring-shaped configuration that is necessary during use of the device. Suitable shape-memory materials include, by way of non-limiting example, a shape-memory metal, such as an alloy of titanium and nickel (e.g., nitinol), that changes its shape upon the application of a force, such as a tension, and that returns to its deployed state upon removal of the force. A person skilled in the art will appreciate that the wire forming the anastomosis device can also be formed from other materials as well. For example, the wire can be formed from a spring material or from a compressible wire material. The device can also be formed from superelastic metal materials, such as alloys of titanium and nickel, that have the ability to undergo a relatively large elastic deformation when mechanically loaded. Additionally, one skilled in the art will understand that the wire can have a variety of cross-sectional shapes and thicknesses or diameters. For example, the wire can have a round, square, or hexagonal shape and can have a thickness or diameter in the range of about 0.008 inches to 0.023 inches.

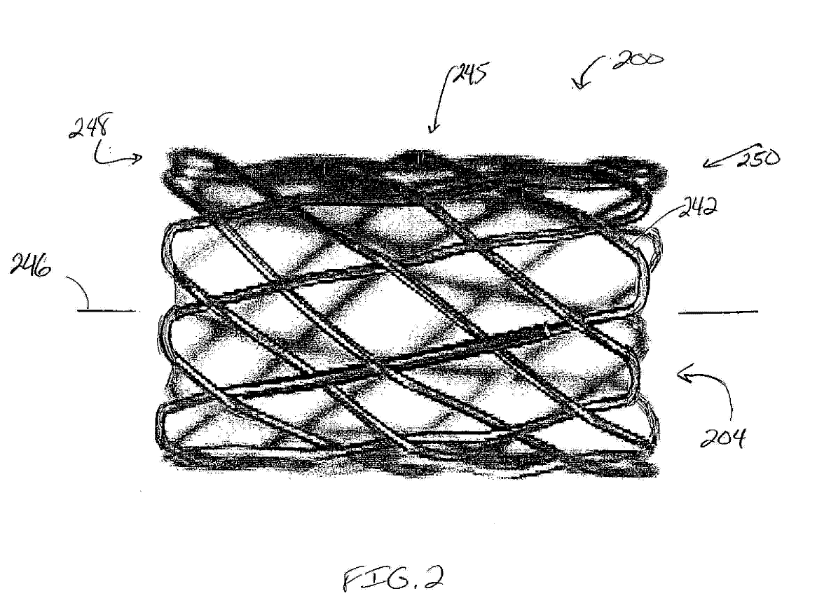

As indicated above, the present invention provides both necrosing and non-necrosing anastomosis devices. FIGS. 2-7 show various exemplary embodiments of anastomosis devices that are adapted to cause necrosis of tissue at an anastomosis site. In general, each device has a ring shaped configuration in a resting or deployed state with an inner and outer periphery. The inner and outer peripheries are configured such that, in the deployed state, pressure is highest at the outer periphery of the deployed device and decreases from the outer periphery to the inner periphery of the device. The relatively high pressure can impede blood flow to the tissue about the outer periphery, thereby causing the tissue inside the outer periphery of the device to become necrotic in this region. As the tissue necroses over time, the device can become separated from the healthy tissue at the anastomosis site and can be passed through the digestive system of the patient.

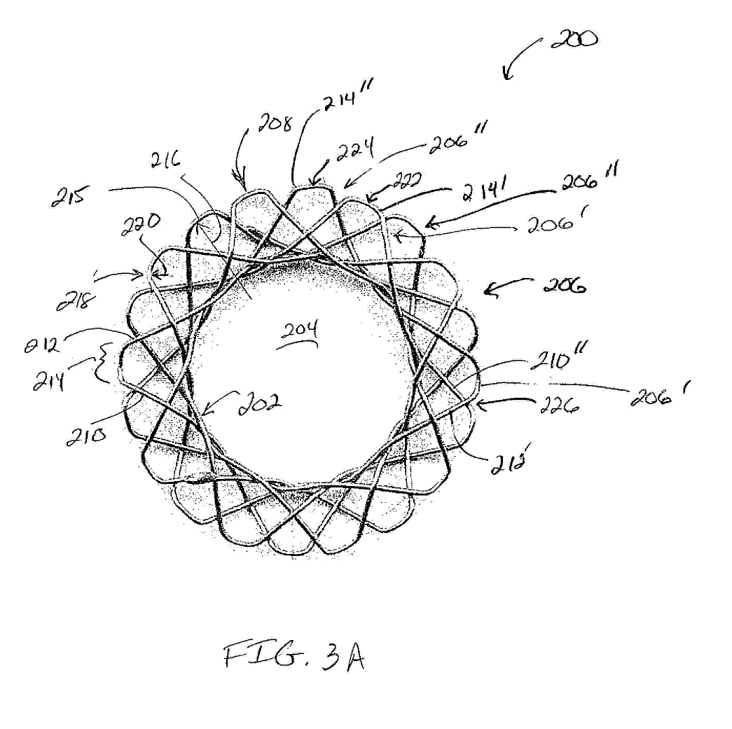

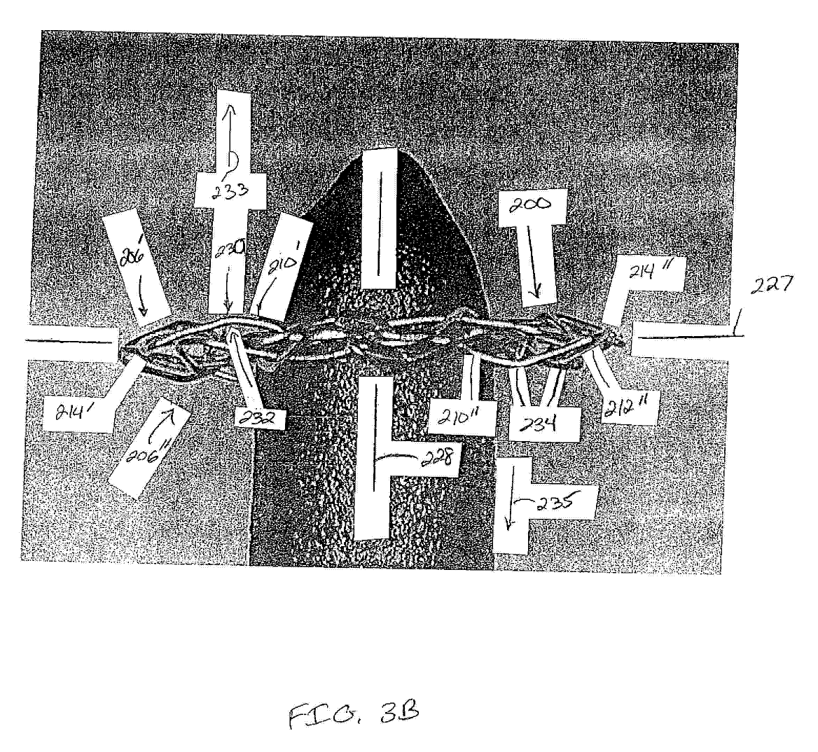

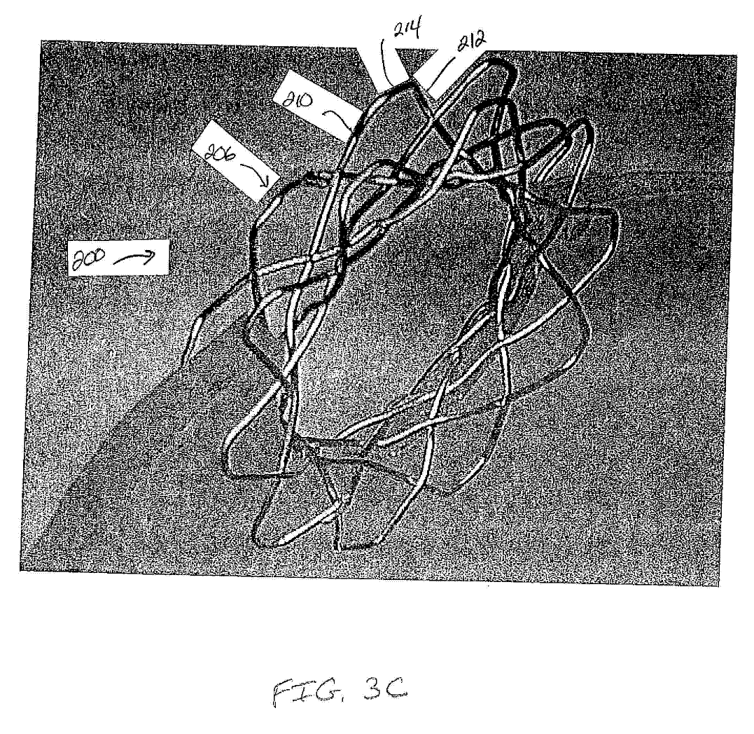

FIG. 2 illustrates one embodiment of an anastomosis device 200 in the expanded elongated tubular configuration, which can be achieved by applying a force, such as a tension, to the device. As shown, the device 200 has a wire mesh wall 242 that includes first 248 and second 250 ends and a midportion 245 disposed between the ends 248, 250. The wire mesh wall 242 also defines a central lumen or opening 204 extending along a central or longitudinal axis 246. The central lumen 204 allows the device 200 to be disposed on the shaft of a delivery device to be delivered to an anastomosis site. As the anastomosis device 200 is deployed from the delivery device, the device 200 will collapse from the elongated state to a resting state to secure opposed tissue layers in apposition. In particular, the wire mesh wall 242 of the tube will contract in the direction of the longitudinal axis 246, the midportion 245 of the device will collapse inward to form an inner periphery of the ring, and ends 248, 250 of the tube will evert to form an outer periphery of the ring. The device 240 will thus have a generally annular configuration in the resting or deployed position, as shown in FIGS. 3A-3C. In particular, the anastomosis device 200 has an annular, ring-shaped configuration with an inner periphery 202 that defines an opening 204 and petals 206 extending from the inner periphery 202 and defining an outer periphery 208. The inner periphery 202 of the device 200 is defined by overlapping of the meshed wire and the outer periphery 208 is defined by the opposed ends of the tube which form opposing superior and inferior petals 206′, 206″. In the deployed state as shown, the inner periphery 202 can maintain a passageway between two apposed tissue layers at an anastomosis site to allow fluid and/or other substances to be passed between the layers and the superior and inferior petals 206′, 206″ can engage and apply pressure to tissue captured therebetween. In an exemplary embodiment, the superior and inferior petals 206′, 206″ can be configured to apply pressure to particular regions of tissue disposed therebetween to facilitate necrosis.

In the embodiment shown in FIGS. 3A-3C, the petals 206 are adapted to apply a pressure to tissue such that the pressure is highest at the outer periphery 208 and decreases from the outer 208 to the inner periphery 202. In particular, each petal 206 can be formed by adjacent arms 210, 212 connected by a tip 214 that extends along a portion of the outer periphery 208 of the device 200. The tip 214 has a bend 215 with a first radius 216, and each arm 210, 212 is connected to the tip 214 at a bend 218 that has a second radius 220. In one aspect, the first radius 216 is greater than the second radius 220. By way of example, the bend 215 can have a radius 216 of approximately 0.44 inches, while the bends 218, which can form transitions between each arm 210, 212 and the tip 214, have a radius 220 of approximately 0.04 inches.

Additionally, as indicated in FIG. 3B, the adjacent arms 210, 212 of each petal 206 can be curved relative to a plane 227 of the device 200 (e.g., in the deployed state) that is substantially perpendicular to a central axis 228 extending through the opening or lumen 204 of the device 200. For example, each of the arms 210′, 212′ forming each of the superior petals 206′ can include an arc portion 230 that extends away from the plane 227 along a first longitudinal direction 233 and that defines an opening 232 that faces (e.g., opposes) the inferior set of petals 206″. Additionally, each of the arms 210″, 212″ forming each of the inferior petals 206″ can include an arc portion 234 that extends away from the plane 227 along a second longitudinal direction 235 and that defines an opening 236 that faces (e.g., opposes) the superior set of petals 206′. The arc portions 230, 232 of the opposed petals 206′, 206″ are configured to orient the respective petal tips 214′, 214″ relative to the plane 227 of the device 200 such that the petal tips 214′, 214″ cross the plane 227 of the device 200 (e.g., where the tips 214′ of the superior petals 206′ extend below the plane 227 and the tips 214″ of the inferior petals 206″ extend above the plane 227 of the device 200). In use, the curved arc to the arms 210, 212 of the petals 206 can help to focus pressure applied by the petals 206 to the outer periphery 208 of the device 200.

The superior and inferior petals 206′, 206″ can also be positioned at particular locations relative to one another to effect tissue necrosis at the outer periphery 208 of the device 200. As shown in FIG. 3A, for example, the tips 214 of the superior and inferior petals 206′, 206″ can be staggered relative to each other about the outer periphery of 208 to distribute pressure to tissue about the circumference or outer periphery 208 of the device 200. In particular, the tips 214′ of each of the superior petals 206′ have midpoints 222 that are staggered or offset from the midpoints 224 of the adjacent tips 214″ of the inferior petals 206″. While the midpoints 222, 224 of adjacent, opposed petals 206′, 206″ can be staggered by any amount, in one embodiment, the midpoints 222, 224 are staggered by an amount in the range of about 15° to 20°, and more preferably by about 18°. As further illustrated in FIG. 3A, the petals 206 are configured such that the tips 214 of opposed, adjacent superior and inferior petals 206′, 206″ do not cross each other, however the arms 210, 212 forming each of the petals 206 are configured to cross at multiple locations about the circumference of the device 200. For example, an arm 212′ of a superior petal 206′ crosses an arm 210″ of an inferior petal 206″ to form a crossing location 226. When the device 200 is deployed at an anastomosis site, the arms 210, 212 of the opposing petals 206 can contact or clamp tissue disposed between the opposed petals 206 at the crossing locations 226 and can apply a relatively large pressure to tissue at the locations 226 while each tip 214 is able to contact and apply pressure to the tissue disposed between adjacent locations 226 to limit or prevent blood flow thereto and to cause the tissue to become necrotic.

The number and size of petals 206 can also vary to obtain a desired result. In the embodiment illustrated in FIGS. 3A-3C, the device 200 includes ten superior petals and ten inferior petals. However, other numbers of petals 206 can be used. For example, a device can be formed with fewer superior and inferior petals 206′, 206″ to increase the stiffness of the device 200 and thus increase the amount of pressure applied by the device 200 on tissue at the outer periphery of the device 200. In another example, a device can be formed with additional superior and inferior petals 206′, 206″ to decrease the stiffness of the device 200 and thus decrease the amount of pressure applied by the device 200 on tissue at the outer periphery of the device 200.

The anastomosis devices described herein can be deployed using a delivery device of the type known in the art, such as that described in U.S. Patent Application Publication No. 2003/0120292, which is hereby incorporated by reference. In use, such as in a side-to-side intestinal anastomosis, the anastomosis device 200 can be expanded into an elongate tubular configuration, such as illustrated in FIG. 2, and disposed about a delivery device. The delivery device can then be inserted within a patient and advanced intraluminally to an anastomosis site. At the anastomosis site, two tissue layers, such as a tissue wall forming an upper stomach pouch and a tissue wall forming a portion of the patient's small intestine, can be brought into apposition. Openings can then be formed within the walls of the tissue such as by a retractable cutting instrument associated with the delivery device. The anastomosis device 200 can then be deployed from the delivery device at the juncture of the apposed openings such that the device 200 collapses from the elongated state to the resting, annular, ring-shaped configuration with the superior petals 206′ and the inferior petals 206″ disposed on opposite sides of the two tissue layers.

Once the anastomosis device 200 has collapsed from the elongated to the resting state, opposing arms 210, 212 of adjacent petals 206 apply a relatively large pressure to the tissue disposed between the superior and inferior petals 206′, 206″ at locations 226, thereby limiting or preventing blood from flowing to these tissue regions. Additionally, the tips 214 of each petal 206 can apply a pressure to the tissue disposed at the outer periphery 208. For example, each tip 214 can apply a load to the tissue disposed between adjacent locations 226 at the outer periphery 208 to limit or prevent blood from flowing to the tissue disposed at the outer periphery 208 of the device 200. Over time, as a result of the pressure applied by the petals 206 on the tissue, the tissue can become necrotic at the outer periphery 208 and inbound of the outer periphery 208, thereby allowing the device to become separated from the healthy tissue at the anastomosis site and passed through the digestive system of the patient.

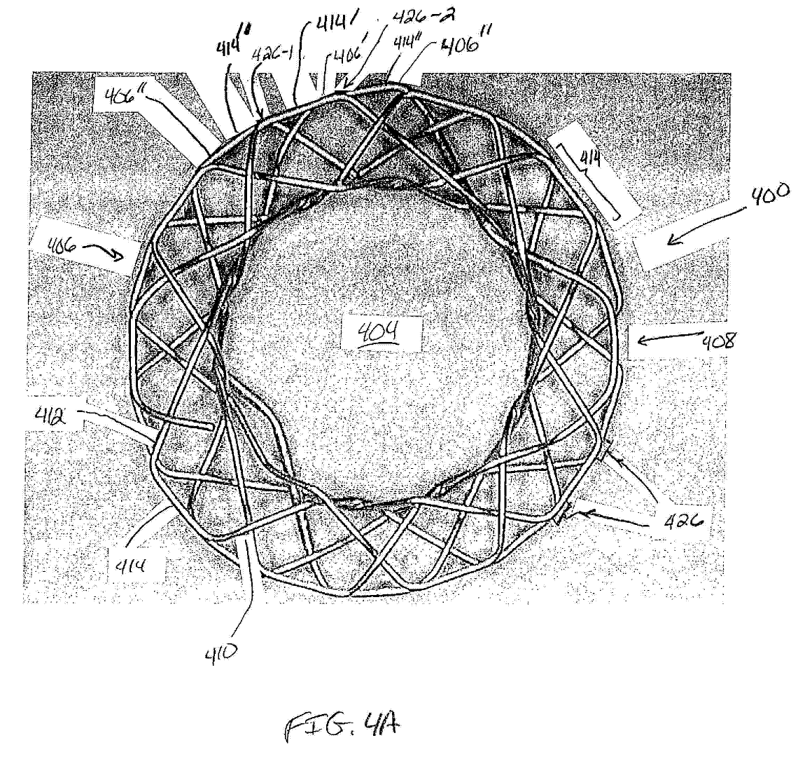

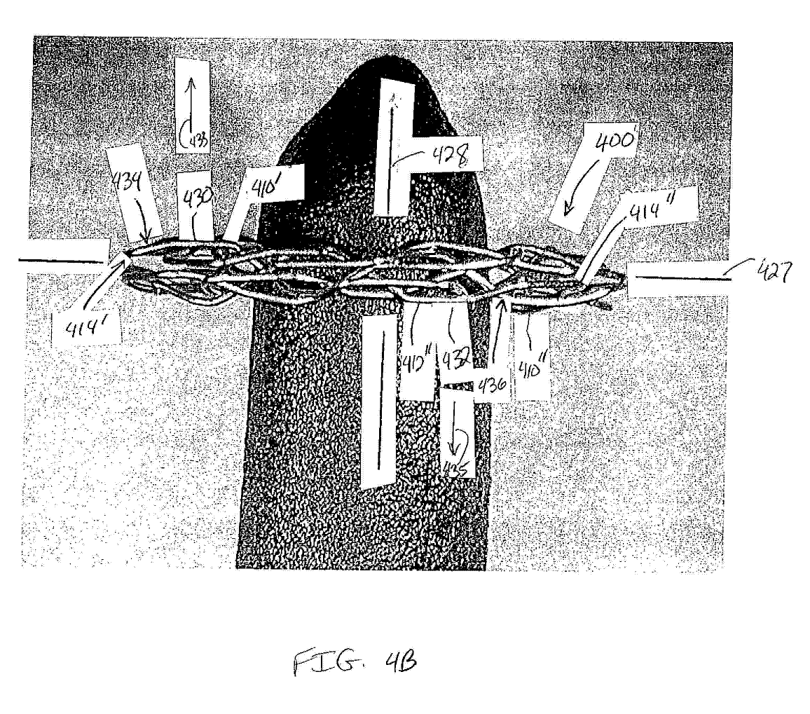

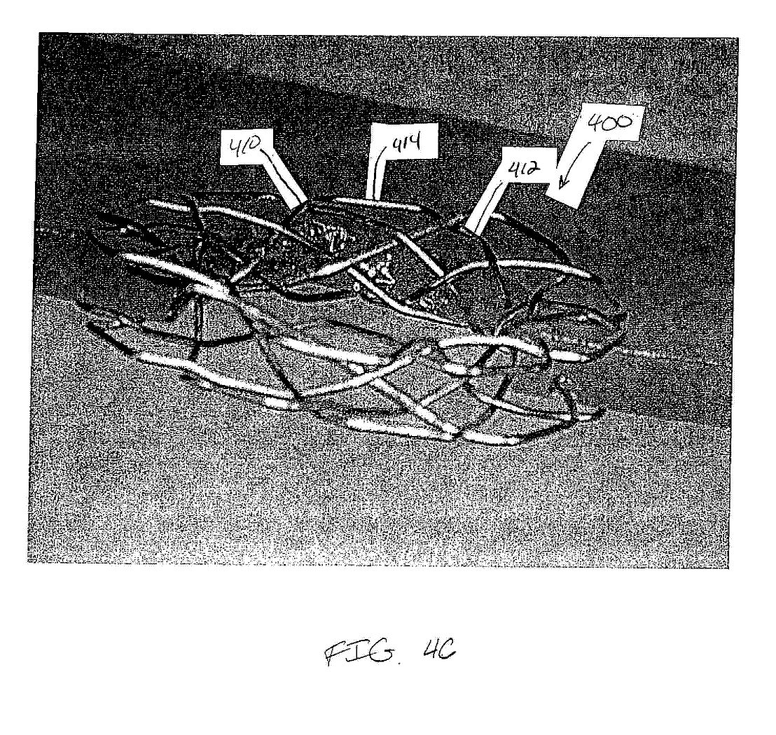

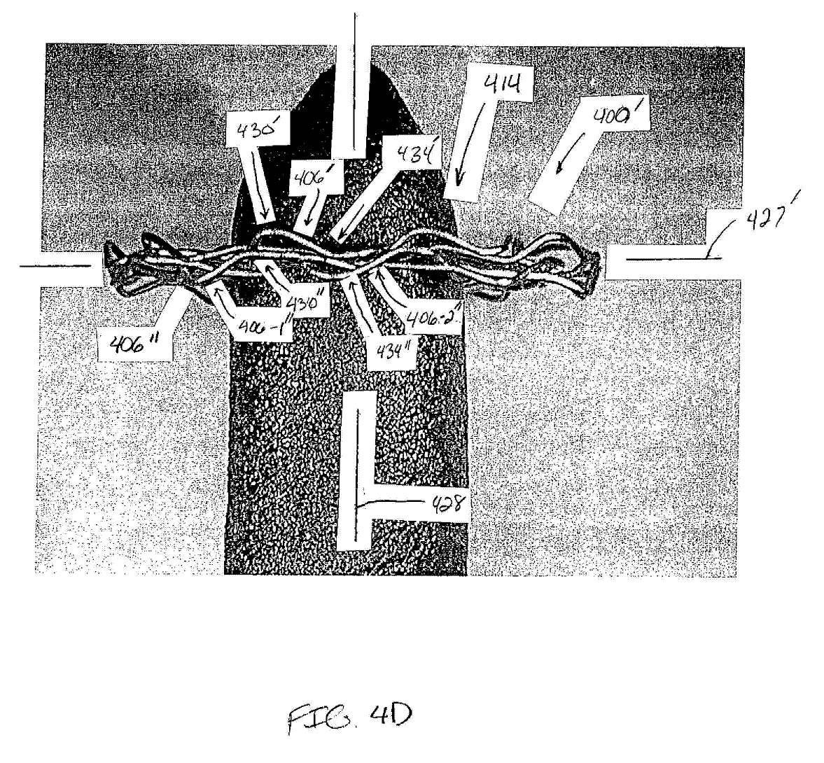

FIGS. 4A-4C illustrate another embodiment of an anastomosis device 400, in the resting state, that is adapted to necrose tissue. In this embodiment, each petal 406 can be formed by adjacent arms 410, 412 connected by a tip 414 that extends along a portion of the outer periphery 408 of the device 400. As illustrated in FIGS. 4B and 4C, the adjacent arms 410, 412 of each petal 406 can be curved relative to a plane 427 of the device 200. For example, each of the arms 410′, 412′ of the superior set of petals 406′ can include an arc portion 430 that extends away from the plane 427 in a first longitudinal direction 433 and a bend portion 434 that orients the tip 414′ substantially parallel to the plane 427 of the device 400. Additionally, each of the arms 410″, 412″ of the inferior set of petals 406″ can include an arc portion 434 that extends away from the plane 227 in a second longitudinal direction 435 and a bend portion 436 that orients the tip 414″ substantially parallel to the plane 427 of the device 400. In use, the curved arc of the arms 410, 412 of the petals 406 can focus pressure applied by the petals 206 to the outer periphery 408 of the device 400.

The superior and inferior petals 406′, 406″ can also be positioned at particular locations relative to one another to facilitate necrosis formation at the outer periphery 408. As shown in FIG. 4A, opposed petals 406 are staggered relative to each other and portions of the tips 414 of adjacent petals 406 are configured to cross at multiple locations 426 about the circumference of the of the device 400. For example, each tip 414′ of superior petal 406′ crosses the tips 414″ of two adjacent inferior petals 406″ at locations 426-1, 426-2. Once deployed in tissue, opposing tips 414 can contact (e.g., clamp) tissue at each of the locations 426 and can apply a relatively large pressure (e.g., localized pressures calculated to be in the range of approximately 40 psi to 70 psi) to the tissue to limit or prevent blood flow thereto and to cause the tissue to become necrotic at the outer periphery 408 and inbound of the outer periphery 408. Also, the amount of overlap of the tips 414, relative to a circumference of the device 400, can affect the degree of tissue necrosis at the outer periphery 408. For example, in the embodiment of FIGS. 4A-4C, opposing petal tips 414 can overlap and clamp tissue along approximately 40% of the outer periphery 408 or circumference of the deployed device 400 and, as a result, can cause substantially uniform necrosis of the tissue about the outer periphery 408, thereby allowing the anastomosis device 400 as well as the necrotic tissue to separate from the healthy tissue and pass through the digestive system of a patient.

One skilled in the art will understand that the tips 414 of the petals 406 can be configured in a variety of different ways. In one embodiment of the device 400′, as shown in FIG. 4D, the tips 414 of the petals 406 can be arced or curved relative to the plane 427 of the device 400′. For example, the tip 414 of each of the superior and inferior petals 406′, 406″ has a peak portion 430 that defines an inferior facing opening and a trough portion 434 that defines a superior facing opening. With the opposed superior and inferior petals 406′, 406″ petals being staggered relative to each other, the peaks 430 and troughs 434 of opposed superior and inferior petals 406′, 406″ can be aligned along the outer periphery. For example, a peak portion 430′ of the superior petal 406′ can be aligned with a peak portion 430″ of a first inferior petal 406-1″ and a trough portion 434′ of the superior petal 406′ can be aligned with a trough portion 434″ of a second inferior petal 406-2″. The overlapping peaks 430 and troughs 434 of the opposed petals 406 form a relatively long circumferential path about the outer periphery 408. As such, tissue disposed at the outer periphery 408 can become stretched by the overlapping peaks 430 and troughs 434 of the opposed petals 406 and, as a result, the thickness of the affected tissue will decrease. Such a decrease in tissue thickness can result in the tissue being exposed to relatively high pressures as applied by the device 400. In use, the overlapping peaks 430 and troughs 434 of opposed petals 406 can clamp tissue along approximately 50% of the outer periphery 408 of the device 400 and can apply a relatively large force (e.g., localized pressures calculated to be in the range of about 75 to 80 psi) to tissue disposed at the outer periphery 408 to cause substantially uniform necrosis of the tissue.

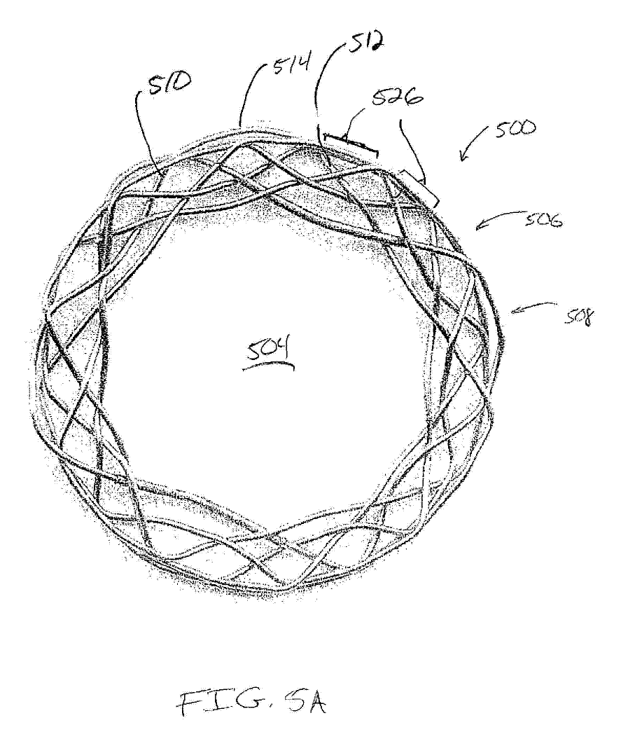

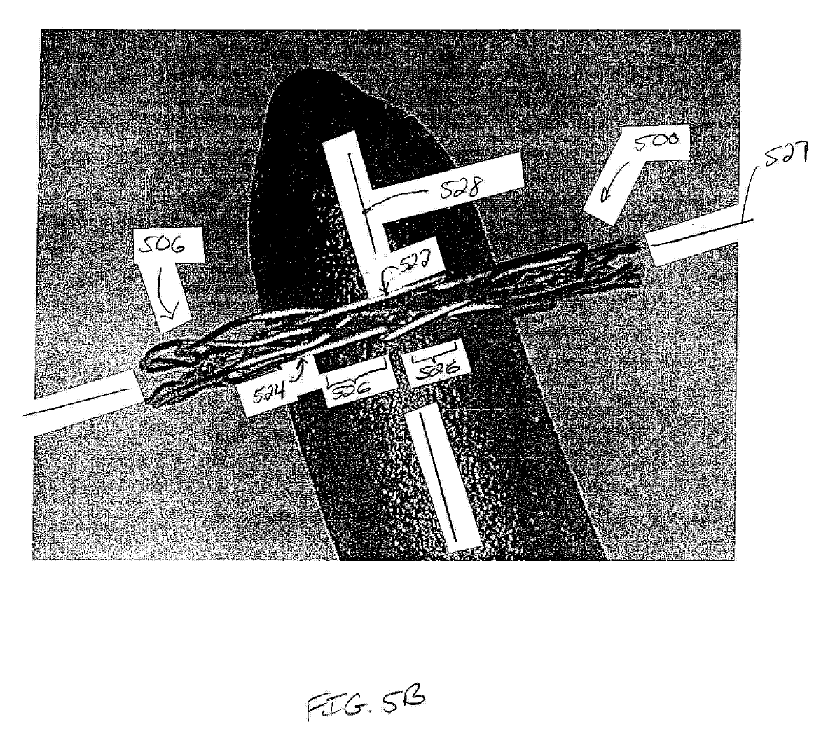

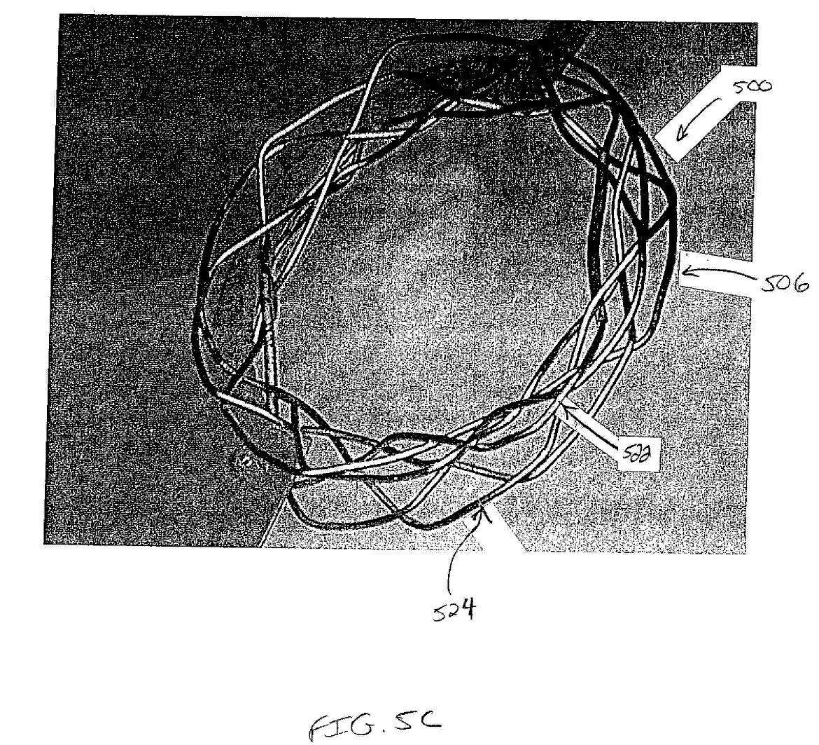

FIGS. 5A-5C illustrate an embodiment of an anastomosis device 500, in the resting state, having opposed petals 506 in which the adjacent arms 510, 512 and tip 514 of each petal 506 are oriented in a substantially planar configuration and where the petal tips 514 of the opposed petals 506 have midpoints 522, 524 that are staggered or offset relative to each other. As shown in FIG. 5B, the arms 510, 512 and tips 514 are oriented to be substantially parallel to a plane 527 that is perpendicular to a central axis 528 of the device 500. Further, the tips 514 of the opposing petals 506 cross at multiple locations 526 about an outer periphery 508 of the device 500. By way of example, the crossing portions 526 of the tips 514 can overlap and clamp tissue along approximately 85% of the outer periphery 508 of the deployed device 500.

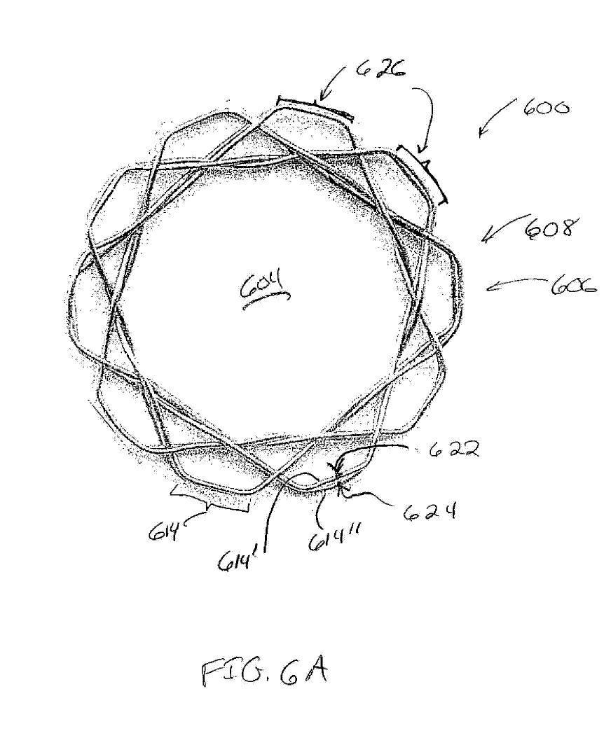

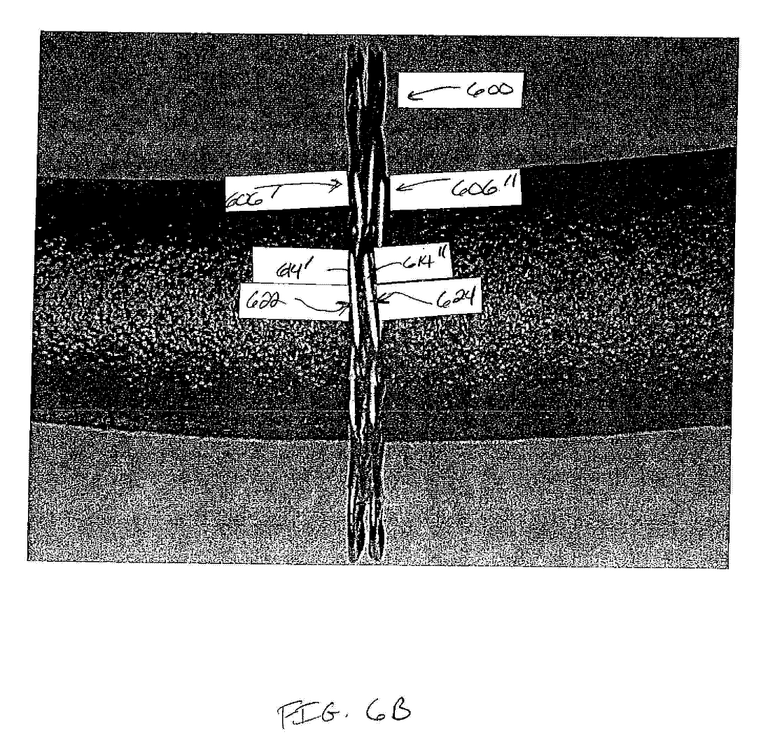

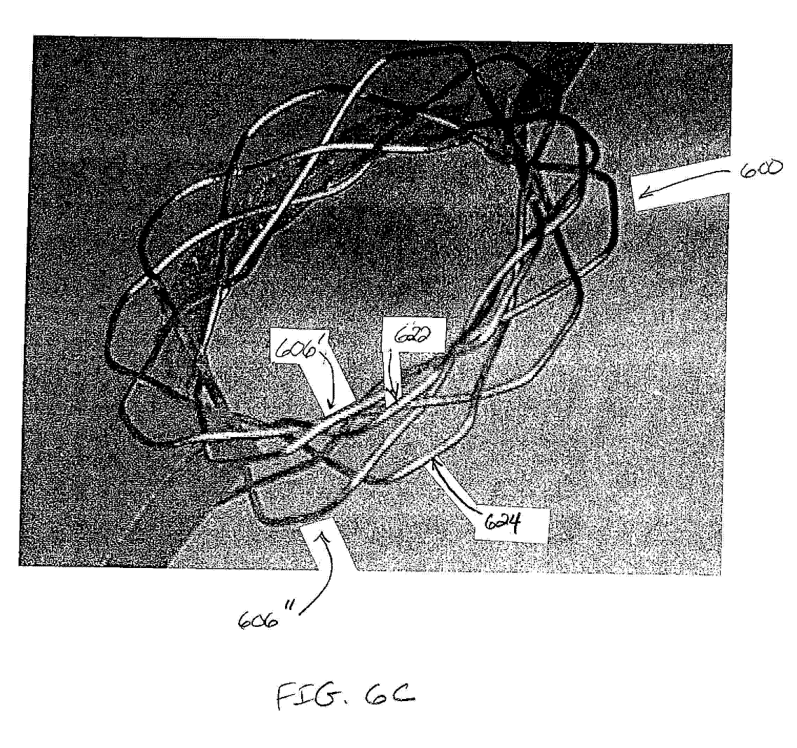

FIGS. 6A-6C illustrate another embodiment of an anastomosis device 600 that is adapted to necrose tissue. In this embodiment, opposed petals 606 are substantially aligned with each otherabout an outer periphery 608 of the device 600. That is, the tips 614′ of each of the superior petals 606′ have midpoints 622 that are substantially aligned with midpoints 624 of the tips 614″ of the inferior petals 606″. As a result, the tips 614 of the aligned superior and inferior petals 406′, 406″ cross at multiple locations 626 about the outer periphery 608 of the of the device 600 to clamp tissue along approximately 77% of the outer periphery 608. In use, opposing tips 614′, 614″ can clamp tissue at each of the locations 626 to apply a relatively large pressure (e.g., localized pressures calculated to be in the range of about 20 to 25 psi) to the tissue to limit or prevent blood flow thereto and to cause the clamped tissue to become necrotic.

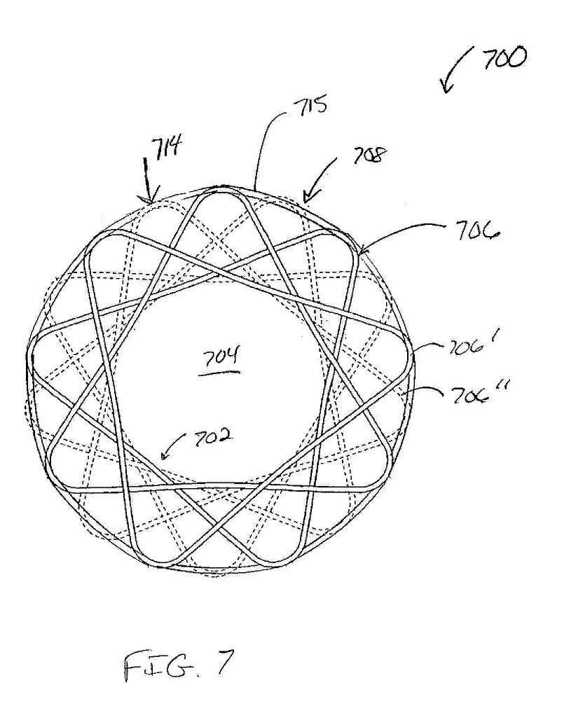

FIG. 7 illustrates another embodiment of an anastomosis device 700, in a resting state, that is adapted to necrose tissue. As shown, the anastomosis device 700 has an annular, ring-shaped configuration with an inner periphery 702 that defines an opening 704 and petals 706 extending from the inner periphery 702 and defining an outer periphery 708. The inner periphery 702 of the device 700 is defined by overlapping of the meshed wire and the outer periphery 708 is defined by the tips 714 of the superior and inferior petals 706. The outer periphery 708 of the anastomosis device 700 also includes opposing pressure distribution rings 715 disposed about to the tips 714 of each set of the opposed petals 716. For example, the device 700 includes an upper ring 715 disposed about the tips 714 of the superior petals 706′ and a lower ring (not shown) disposed about the tips 714 of the inferior petals 706″. In use, the pressure distribution rings 715 can distribute pressure from the petal tips 714 to tissue disposed substantially along the entire outer periphery 708 of the device 700 to create a substantially uniform blood flow barrier about the outer periphery 708 of the device 700. For example, the anastomosis device 700 can clamp tissue along approximately 90% of the outer periphery 708 to apply a substantially uniform pressure of approximately 35 psi to the tissue disposed at the outer periphery 708.

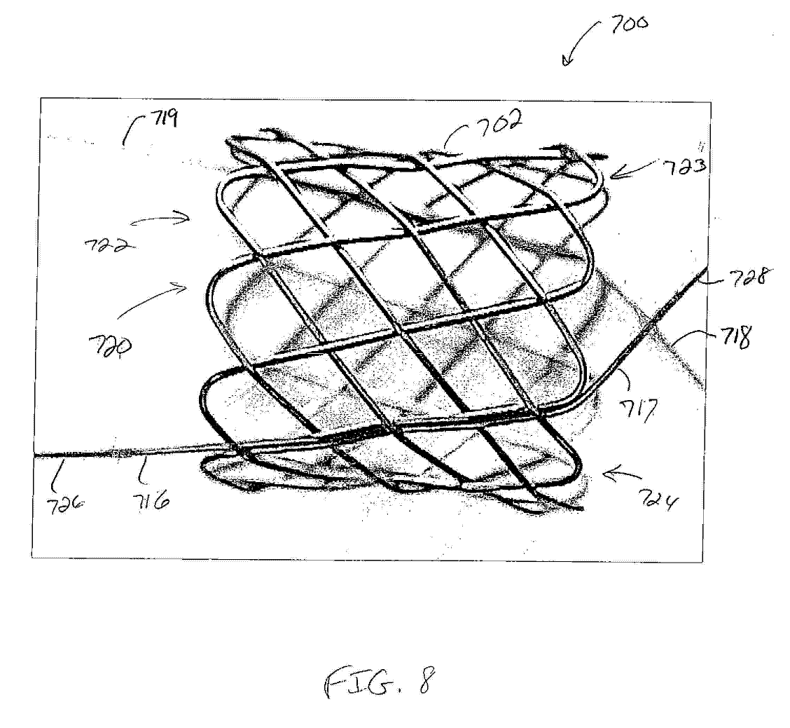

One skilled in the art will understand that the pressure distribution rings 715 can be formed at the outer periphery 708 of the device 700 in a number of ways. For example, as described below with respect to FIG. 8, when the anastomosis device 700 is manufactured, two wires can be woven together to form a mesh wall 702 such that, at the end of the weaving process, portions 716, 717, 718, 719 of the two wires can extend from the device 700. Two of the end wires can then be removed from the device 700 while the remaining two end wires can be used to form the pressure distribution rings 715. For example, the wire portions 718 and 719 can be removed from the mesh wall 702, the wire portion 716 can be interwoven with the tips 720 of a first end 722 of the device 700 to form a first pressure distribution ring, and the wire portion 719 can be interwoven with the tips 723 of a second end 724 of the device 700 to form a second pressure distribution ring. Once the wire portions 716, 719 have been woven to form the pressure distribution rings, the respective free ends 726, 728 of the wire portions 716, 719 can remain uncoupled to the mesh wall 702. In use, as the device 700 is deployed from an applier and collapses from an expanded, tubular shape to an annular, deployed shape, the free ends 726, 728 of the wire portions 716, 719 can slide through the tips 720, 723 to allow the pressure distribution rings 715 of the device 700 to expand from a collapsed state to an expanded state. When deployed, the pressure distribution rings 715 can distribute pressure from the petal tips 714 to tissue disposed substantially along the entire outer periphery 708 of the device 700.

While the pressure distribution rings 715 can be formed at the outer periphery 708 of the device 700 using two of the wire portions 716, 717, 718, 719 that extend from the device 700, one skilled in the art will understand that the pressure distribution rings 715 can be formed from separate wires that are added to the device 700. For example, at the end of the weaving process, all of the wire portions 716, 717, 718, 719 can be removed from the device 700 and separate wire elements can be attached. In particular, wire elements having a larger or smaller diameter than that of the wire forming the mesh wall 702 or wire elements formed of a material that is different than the wire forming the mesh wall 702 can be attached to the device 700.

The various anastomosis devices described above are adapted to cause necrosis of the clamped tissue. It is sometimes desirable, however, for an anastomosis device to allow tissue to overgrow the device at an anastomosis site. A non-necrosing anastomosis device is similar to necrosing anastomosis devices in that it has a ring shaped configuration in a resting or deployed state with an inner and outer periphery.

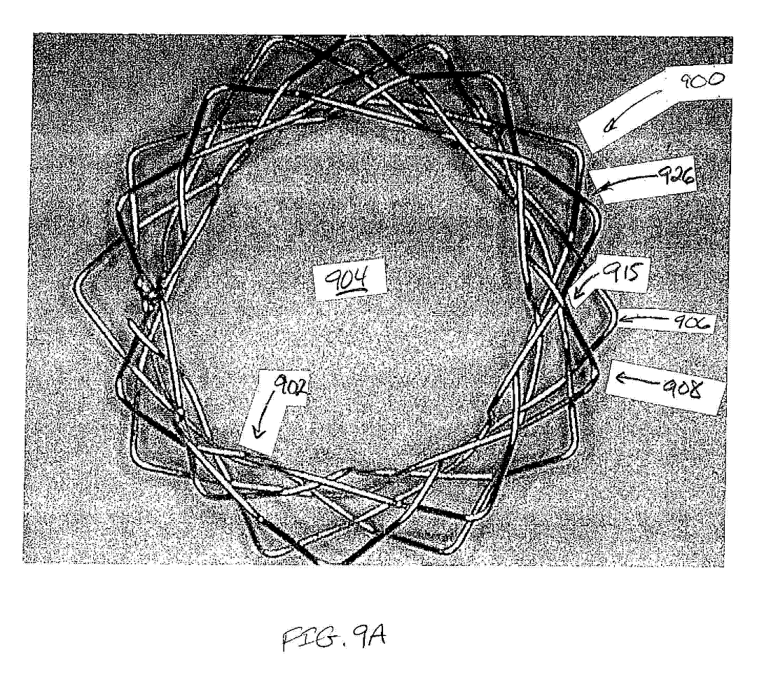

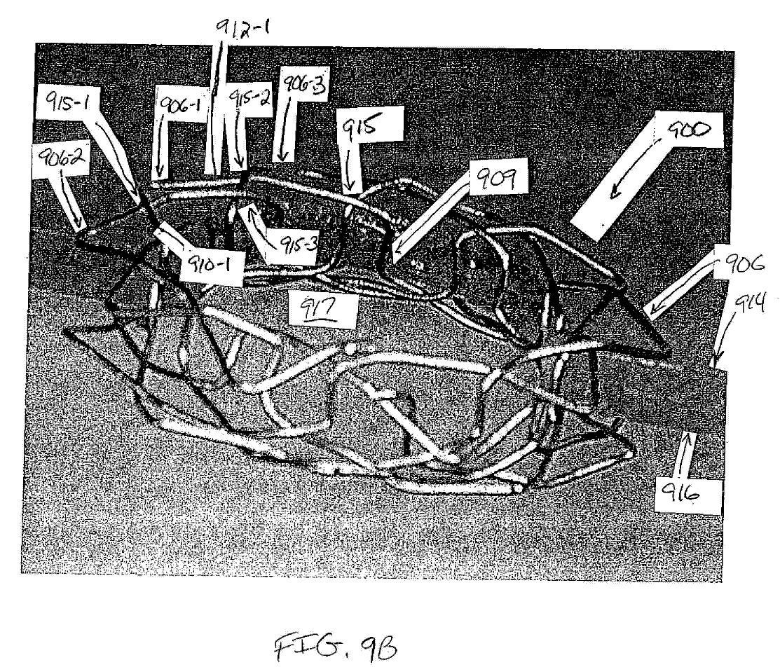

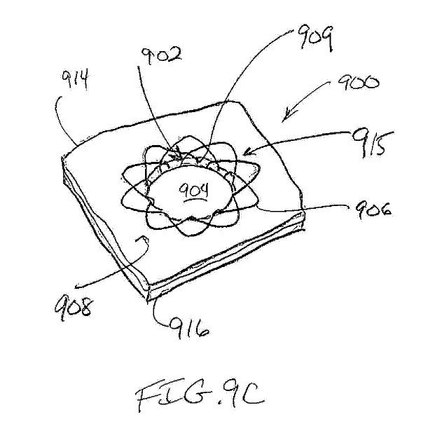

FIGS. 9A-9C show one embodiment of a non-necrosing type anastomosis device 900 in a resting or deployed state. As shown, the anastomosis device 900 has an annular, ring-shaped configuration with an inner periphery 902 that defines an opening 904 and petals 906 extending from the inner periphery 902 and defining an outer periphery 908. One characteristic of such a non-necrosing anastomosis device 900 is that the inner periphery 902 of the device 900 is formed from nonoverlapping wire segments 909. For example, as illustrated in FIG. 9B, the device 900 can be woven such that in the deployed state the wire segments 909 disposed at the inner periphery 902 of the device 900, within a lumen 917 formed between opposed tissues 914, 916, do not contact each other. Instead, the wires forming the arms of adjacent superior or inferior petals 906 can contact each other at locations 915. For example as illustrated in FIG. 9B, an arm 912-1 of a first superior petal 906-1 is disposed beneath an arm of a second superior petal 906-2 at location 915-1, an arm 910-1 of the first superior petal 906-1 is disposed over an arm of a third superior petal 906-3 at location 915-2, and the arms of the second and third superior petals 906-2, 906-3 contact each other at location 915-3. It is believed that such a design discourages the growth of bacteria or biofilms which can limit or prevent tissue growth due to the lack of contact between wire segments 909 at the inner periphery. Thus, tissue at the inner periphery 902 is able to grow over the wire segments 909. Accordingly, when the device 900 is in the deployed state as shown in FIG. 9C, the wire segments 909 forming the inner periphery 902 maintain a passageway between the two apposed tissue layers 914, 916 at an anastomosis site to allow fluid and/or other substances to be passed between the layers 914, 916 and promote tissue overgrowth of the segments 909 at the inner periphery 902. The device 900 still enables the superior and inferior petals 906 to engage the tissue layers 914, 916 and apply a pressure to the layers 914, 916 that is sufficient to maintain apposition of the tissue layers 914, 916 but that is below a threshold that can cause the tissues to become necrotic. For example, the opposing superior and inferior petals 906′, 906″ can overlap at locations 926 about the outer periphery 908 to apply a pressure of less than about 2 psi to the tissue layers 914, 916 at each of the locations 926.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. An anastomosis device, comprising:
a body formed from a woven shape-memory wire and defining a central lumen, the body being configurable between an expanded position in which the body assumes a generally tubular configuration having a wire mesh wall adapted for insertion into a lumen of an anastomotic site and a rest position in which the body assumes an annular configuration having a plurality of petals with petal tips that define an outer periphery, the rest position being effective to hold opposed tissues of the anastomotic site in apposition and to apply a pressure to the opposed tissues such that the pressure decreases from an outer periphery to an inner periphery of the device.

2. The anastomosis device of claim 1, wherein the plurality of petals are configured to apply the pressure to the opposed tissues to cause necrosis of the opposed tissues in a contact region.

3. The anastomosis device of claim 1, wherein the plurality of petals comprise a superior set of adjacent petals and an inferior set of adjacent petals, and the device is adapted to receive the opposed tissues between the inferior and superior sets of petals.

4. The anastomosis device of claim 3, wherein, in a rest position, each petal of the superior set and inferior set of adjacent petals is formed by adjacent arms connected by a tip that extends along a portion of the outer periphery of the device, the tip having a first radius and each arm being connected to the tip at a bend that has a second radius, the first radius being greater than the second radius.

5. The anastomosis device of claim 4, wherein, when the device is configured in the rest position, the adjacent arms and tip of each petal are oriented in a substantially planar configuration in a plane that is substantially perpendicular to a central axis extending through the central lumen of the device.

6. The anastomosis device of claim 4, wherein the adjacent arms of each petal are oriented in a plane that is substantially perpendicular to a central axis extending through the central lumen of the device, at least a portion of the adjacent arms of the superior set of petals including an arc portion having an inferior facing opening, and at least a portion of the adjacent arms of the inferior set of petals including an arc portion having a superior facing opening.

7. The anastomosis device of claim 6, wherein the adjacent arms of the superior set of petals include a bend portion disposed between the arc portion and the tip, the bend portion of the adjacent arms orienting the tip in the plane that is substantially perpendicular to a central axis extending through the central lumen of the device and wherein the adjacent arms of the inferior set of petals include a bend portion disposed between the arc portion and the tip, the bend portion of the adjacent arms orienting the tip in the plane that is substantially perpendicular to a central axis extending through the central lumen of the device

8. The anastomosis device of claim 4, wherein the tip of each petal has a peak portion defining an inferior facing opening and a trough portion defining a superior facing opening.

9. The anastomosis device of claim 4, wherein the tips of the superior set of adjacent petals have midpoints that are staggered about a circumference of the device relative to midpoints of the tips of the inferior set of adjacent petals.

10. The anastomosis device of claim 4, wherein the tips of the superior set of adjacent petals have midpoints that are substantially aligned with midpoints of the tips of the inferior set of adjacent petals.

11. The anastomosis device of claim 4 wherein the tips of the petals are adapted to contact the opposed tissues along a distance greater than approximately 5% of the circumference of the outer periphery of the device when configured in the rest position.

12. The anastomosis device of claim 3, further comprising:
a first wire circumferentially coupled to the petal tips of the superior set of adjacent petals; and
a second wire circumferentially coupled to the petal tips of the inferior set of adjacent petals, wherein the first wire and the second wire are adapted to contact the opposed tissues along a distance greater than approximately 90% of the circumference of the outer periphery of the device when configured in the rest position.

13. An anastomosis device, comprising:
a body formed from a woven shape-memory wire and defining a central lumen, the body being configurable between an expanded position in which the body assumes a generally tubular configuration having a wire mesh wall adapted for insertion into a lumen of an anastomotic site and a rest position in which the body assumes an annular configuration having a plurality of petals with petal tips that define an outer periphery, the rest position being effective to hold opposed tissues of the anastomotic site in apposition and to allow the petal tips to contact the opposed tissues along a distance greater than approximately 5% of the outer periphery of the device.

14. The anastomosis device of claim 13, wherein the plurality of petals are configured to apply the pressure to the opposed tissues to cause necrosis of the opposed tissues.

15. The anastomosis device of claim 13, wherein the plurality of petals comprise a superior set of adjacent petals and an inferior set of adjacent petals, and the device is adapted to receive the opposed tissues between the inferior and superior sets of petals.

16. The anastomosis device of claim 15, wherein, in a rest position, each petal of the superior set and inferior set of adjacent petals is formed by adjacent arms connected by a tip that extends along a portion of the outer periphery of the device, the tip having a first radius and each arm being connected to the tip at a bend that has a second radius, the first radius being greater than the second radius.

17. The anastomosis device of claim 15, wherein the tips of the superior set of adjacent petals have midpoints that are staggered about a circumference of the device relative to midpoints of the tips of the inferior set of adjacent petals.

18. The anastomosis device of claim 15, wherein the tips of the superior set of adjacent petals have midpoints that are substantially aligned with midpoints of the tips of the inferior set of adjacent petals.

19. An anastomosis device, comprising:
a body formed from a woven shape-memory wire and defining a central lumen, the body being configurable between an expanded position in which the body assumes a generally tubular configuration having a wire mesh wall adapted for insertion into a lumen of an anastomotic site and a rest position in which the body assumes an annular configuration with an inner periphery and having a plurality of petals with petal tips that define an outer periphery, the inner periphery being configured to allow tissue overgrowth of the device at the inner periphery.

20. The anastomosis device of claim 19, wherein, when configured in the rest position, the body forms opposed petals configured to apply a pressure to the tissues less than approximately 2.0 pounds per square inch.

21. The anastomosis device of claim 19, wherein the inner periphery is free of overlying wire segments of opposed superior and inferior portions of the device.

22. A method for anastomosing tissue layers, comprising:
delivering an anastomosis device formed from a woven shape-memory wire having a wire mesh wall defining a central lumen and configured in an expanded, tubular position through a lumen defined by two opposed tissues; and
deploying the anastomosis device to hold the opposed tissues in apposition such that, upon deployment, the device assumes an annular configuration having a plurality of petals with petal tips that define an outer periphery, the device applying a pressure to the opposed tissues such that the pressure decreases from the outer periphery to an inner periphery of the device.
